(19) United States
(12) Plant Patent Application Publication
(10) Pub. No.: US 2020/0296871 P1
(43) Pub. Date: Sep. 17, 2020

(54) Kiwi Plant Named 'RK 2018'

(50) ;

(71) Applicant: Red Kiwi S.r.l., Riolo Terme (IT)

(72) Inventor: Maurizio Dal Pane, Castel Bolognese (IT)

(21) Appl. No.: 16/602,394

(22) Filed: Sep. 27, 2019

(30) Foreign Application Priority Data
Mar. 11, 2019 (QZ) 20190516

(51) Int. Cl. A01H 6/00

(52) U.S. Cl. PLT/156

(57) ABSTRACT

A new and distinct kiwi plant of the species Actinidia chinensis is described. The cultivar results from a controlled pollination using a female A. chinensis selection RS1 and a male selection (Tomuri and Chieftain). Named parents (RS1, Tomuri and Chieftain) are believed to be unpatented cultivars in the United States. The new cultivar is distinguished by its medium fruit size, ovoid fruit shape, general hairlessness, greenish-brown fruit coloring, and its harvest date in mid-October in Italy.

Latin name: Actinidia Chinensis Planch.

Varietal denomination: Red Queen.

### FIELD OF THE INVENTION

Genus and species of plant claimed: Actinidia Chinensis Planch.

### PRIORITY CLAIM

The present application claims priority from Community Plant Variety Office (European Union) Application No. 20190516, filed Mar. 11, 2019, which is herein incorporated in its entirety by reference.

### BACKGROUND OF THE INVENTION

Kiwi plants in cultivation are mainly varieties of A. deliciosa, although some A. chinensis and A. arguta varieties are grown. A. deliciosa and A. chinensis are closely related and varieties of both types have large fruit (about 100 g) with hair on the skin. Main varieties of Kiwi plants include ‘Hayward’ (A. deliciosa) and ‘HORT16A’ (A. chinensis). All Actinidia species are dioecious, so female varieties have to be interplanted with male pollinizers to ensure fruit production. Fruit are usually cut and eaten with a spoon. Tomuri and Chieftain are both esaploid males which are commonly used to pollinate esaploid varieties such as Hayward, and also RS1. As they are both not synchronized with Red varieties, which bloom much earlier than Tomuri and Chieftain, so male pollen has to be collected from male vines one year in advance, and then distributed on females during blooming, either dry (mixed with lycopodium) or in a liquid solution.

RS1 is a red kiwifruit variety which germinates in Northern Italy around 15 days before Hayward (means around 3^{rd} week of March) and blooms 15 days before Hayward (means around April 20^{th}). It is a medium vigorous plant with short interno-knots. It adapts to a wide range of climates, but barely stand temperatures below 0° C. cause of its high sensitiveness to Pseudomonas syringae pv.actinidiae (PSA), a bacteria that can result in the death of kiwifruit vines. It is harvested in Northern Italy about 35 days before Hayward, which means the last week of September.

Fruits can be stored for 2-3 months at 0° C.

### SUMMARY OF THE INVENTION

The present invention is a new and distinctive kiwifruit variety, designated RK 2018. It was obtained during the course of a planned plant breeding program, which took place in the Emilia-Romagna region near Milan, Italy between 2008-2013. The RK 2018 variety was selected from a population of seedlings obtained from freely-pollinated RS1 fruits. About 450 seedlings of RS1 fruits were pollinated with various males, particularly Tomuri and Chieftain, which both proved to be perfectly compatible with RS1. RK 2018 first flowered in April 2011 and fruits were assessed in October 2011. All referenced dates are northern hemisphere timings.

In 2012, RK 2018 variety was grafted on Hayward and Tomuri rootstock, and the asexually reproduced plants proved to be true to type.

In 2014, the first trial plot of plants grafted on both Hayward and Tomuri were established in Latina, Italy. The resulting first fruits harvested in 2016 proved to maintain the unique features observed in the nursery.

Germination period is 10-12 days before Hayward, which means about one week after RS1. Flowering period is last week of April, means 10 days before Hayward and one week after RS1. Ripening period is last week of October, about 25 days after RS1. Harvesting should be performed when the external pulp tends to lose its green color and starts to turn to yellow.

RK 2018 is moderately vigorous and has close internodes. The germination percentage during spring is very high: 70-85%. The plant has a bushy growing habit and responds effectively to cuts of green pruning, generating a large number of offshoots. The RK 2018 variety presents panicles with a number of flowers ranging from 2 to a maximum of 5. The plant is malleable and responds very well to pruning. Production is uniform, with many single fruits, a few double fruits and a high share of Category 1 fruits.

Fruits are oblong, of variable length between 50-80 mm and width between 35-45 mm. The fruit's medial section is circular.

The peel has a green-brown shade of color and is glabrous. The pulp is yellow on the outside and red in the center. The red and yellow color increases in uniformity after the 4th year of implantation and so does the overall ripening of fruits. The fruit's columella is cream-white in color and of a medium-soft consistency, more consistent in proximity of the stalk.

The shelf life of RK 2018 is strongly dependent on the harvesting method, but is typically 4-5 months and, in ideal conditions, it can even reach 6 months.

Brix degrees at the time of consumption range from 18° to 24° with a medium level of acidity.

Distinctive characteristics of the variety include the following shown in Table 1:

| Number | Characteristics | State-Expression |
|---|---|---|
| 1 | Plant: sex | Feminine |
| 2 | Plant: parthenocarpy | Absent |
| 3 | Plant: Vigour | Medium |
| 4 | Young shoot: hair density | Limited |
| 5 | Young shoot: anthocyanin | Limited |
| | coloration on the growth edge | |
| 6 | Stalk: thickness | Medium |
| 7 | Stalk: offshoot color on the | Light green |
| | sun-exposed side | |
| 8 | Stalk: bark texture | Slightly coarse |
| 9 | Stalk: hair density | Light |
| 10 | Stalk: lenticels size | Small |
| 11 | Stalk: lenticels number | Medium |
| 12 | Stalk: surface of the bud's base | Medium |
| 13 | Stalk: presence of pearls | Absent |
| 14 | Stalk: size of the pearl's orifice | Absent |
| 15 | Leaf callus | Concave |
| 16 | Stalk: fibrous part | Fibrous, watery |
| 17 | Leaf blade: shape | Heart-shaped in the |
| | | young age, more |
| | | rounded in the adult |
| | | age |
| 18 | Leaf blade: length/width ratio | 1:1 |
| 19 | Leaf blade: tip's shape | Lanceolate in the |
| | | young age, rounded |
| | | in the adult age |
| 20 | Leaf blade: basal lobes | Lightly overlapping |
| 21 | Leaf blade: number of ciliated | Medium |
| | indentations | |
| 22 | Leaf blade: hair density in | Absent |
| | the upper part | |
| 23 | Leaf blade: hair density in | Very sparse-subtle |
| | the lower part | |
| 24 | Leaf blade: green intensity in | Intense green |
| | the upper part | |
| 25 | Leaf blade: color in the lower part | Light green |
| 26 | Leaf blade: variegation | Absent |
| 27 | Leaf blade: variegation color | Absent |
| 28 | Leaf: stalk length | Long |
| 29 | Stalk: anthocyanin color in | Absent |
| | the upper part | |
| 30 | Panicle: type | Singular |
| 31 | Panicle: number of flowers | 4-7 |
| 32 | Flower bud: first bud position | Basal |
| 33 | Flower: number of sepals | High |
| 34 | Flower: sepal main color | Green |
| 35 | Flower: density of sepal hairs | Medium |
| 36 | Flower: diameter | Medium |
| 37 | Flower: petal placement | Overlapping |
| 38 | Flower: profile shape | Flat |
| 39 | Flower: number of stigma | Medium |
| 40 | Flower: placement of stigma | Irregular |
| 41 | Petal: main color of the | White-yellowish |
| | adaxial side | |
| 42 | Petal: main color shade | Regular |
| 43 | Petal: secondary color on | Green |
| | adaxial side | |
| 44 | Petal: secondary color | Almost absent |
| | distribution | |
| 45 | Anther: color | Intense yellow |
| 46 | Fruit: weight | Average weight: |
| | | 85-90 gr |
| 47 | Fruit: length | 50-80 mm |
| 48 | Fruit: width | 35-45 mm |
| 49 | Fruit: length-width ratio | 1:1.7 |
| 50 | Fruit: shape | Ovoid |
| 51 | Fruit: shape in central section | Circular |
| | (approx. mid fruit) | |
| 52 | Fruit: lower part | Lightly concave |
| 53 | Fruit: presence of sharp | Absent |
| | protuberance | |
| 54 | Fruit: presence of ring at the calyx | Absent |
| 55 | Fruit: edge shape at stem end | Lightly rounded |
| 56 | Fruit: stem length | Long |
| 57 | Fruit: stem length in relation to | Long |
| | fruit length | |
| 58 | Fruit: presence of lenticels on skin | Limited |
| 59 | Fruit: Absence of hair on skin | Hairs sparsely present |
| 60 | Fruit: hair density | Very sparse |
| 61 | Fruit: hair color | Light brown |
| 62 | Fruit: hair adherence to skin | Strong |
| 63 | Fruit: skin color | Green-brown |
| | | depending on |
| | | light exposure |
| 64 | Fruit: hair adherence to pulp | Strong |
| 65 | Fruit: color of external pericarp | Yellowish tending |
| | | to green |
| 66 | Fruit: color of loculi-alveoli | Red |
| 67 | Fruit: diffusion of red color around | High |
| | the loculi-alveoli (if present) | |
| 68 | Fruit: intensity of the red color in | Strong |
| | the loculus | |
| 69 | Fruit: extent of the columella in | Medium |
| | relation to the fruit | |
| 70 | Fruit: overall shape of the | Circular |
| | columella in the mid-section | |
| 71 | Fruit: color of the columella | Cream white |
| 72 | Fruit: sweetness | High |
| 73 | Fruit: acidity | Medium |
| 74 | Period of bud breakage | Medium-premature |
| 75 | Period of flowering | Averagely premature |
| 76 | Period of ripening for harvesting | Averagely tardive |

The new variety can be asexually reproduced, both by promoting roots on cuts, using warm beds, or more conveniently through grafting on selected rootstocks. Plants grafted on male rootstocks proved to be more vigorous and more resistant than straight plants from cuts. Grafting can be operated within a wide window, from May into July. English single or double cut is the most common grafting technique. Trial plantings of grafted plants established in both Emilia Romagna (Castelbolognese) and Latina area in 2014 have shown that the unique combination of characteristics come true to form, are established, and transmitted through succeeding asexual propagations.

### BRIEF DESCRIPTION OF THE DRAWINGS

Images show in the following identified pictures are from plants that are approximately four years from grafting.

FIG. 1 shows typical fruit of the new variety in the orchard.

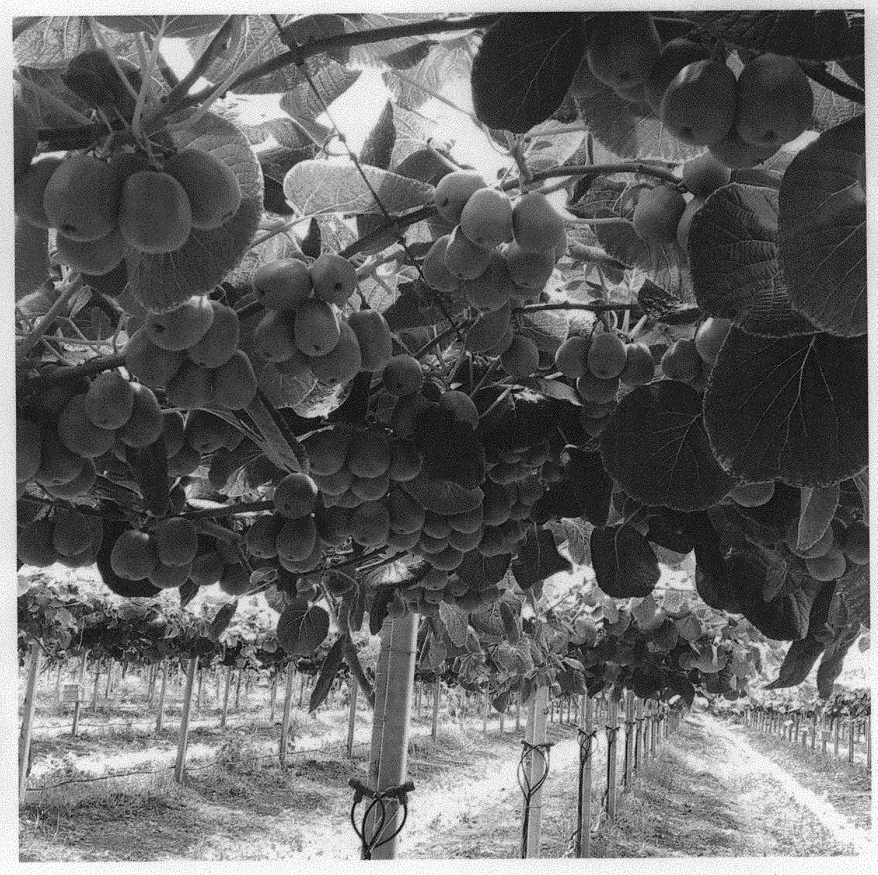

FIG. 2 shows closeup of typical fruit of the new variety in the orchard.

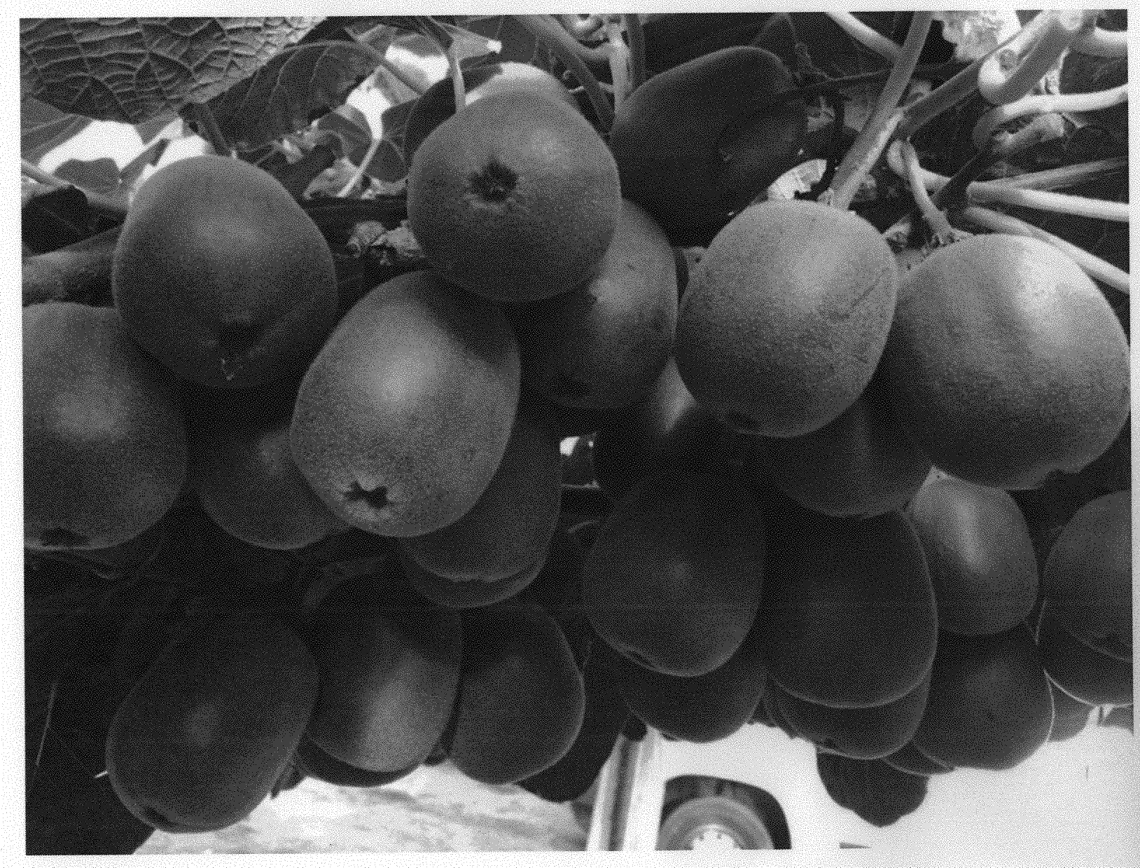

FIG. 3 shows typical fruit of the new variety in the orchard in cross-section, 10 days before harvest.

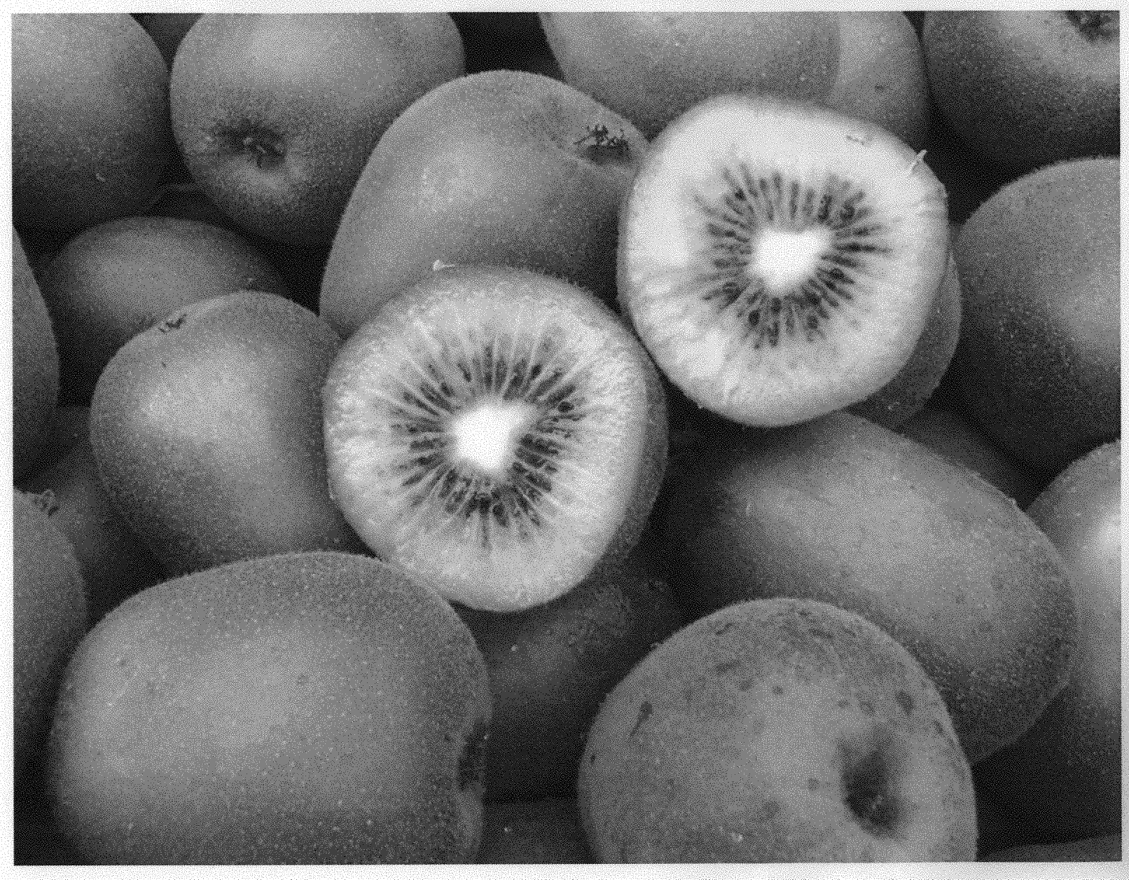

FIG. 4 shows a typical fruit of the new variety in the studio and in cross-section.

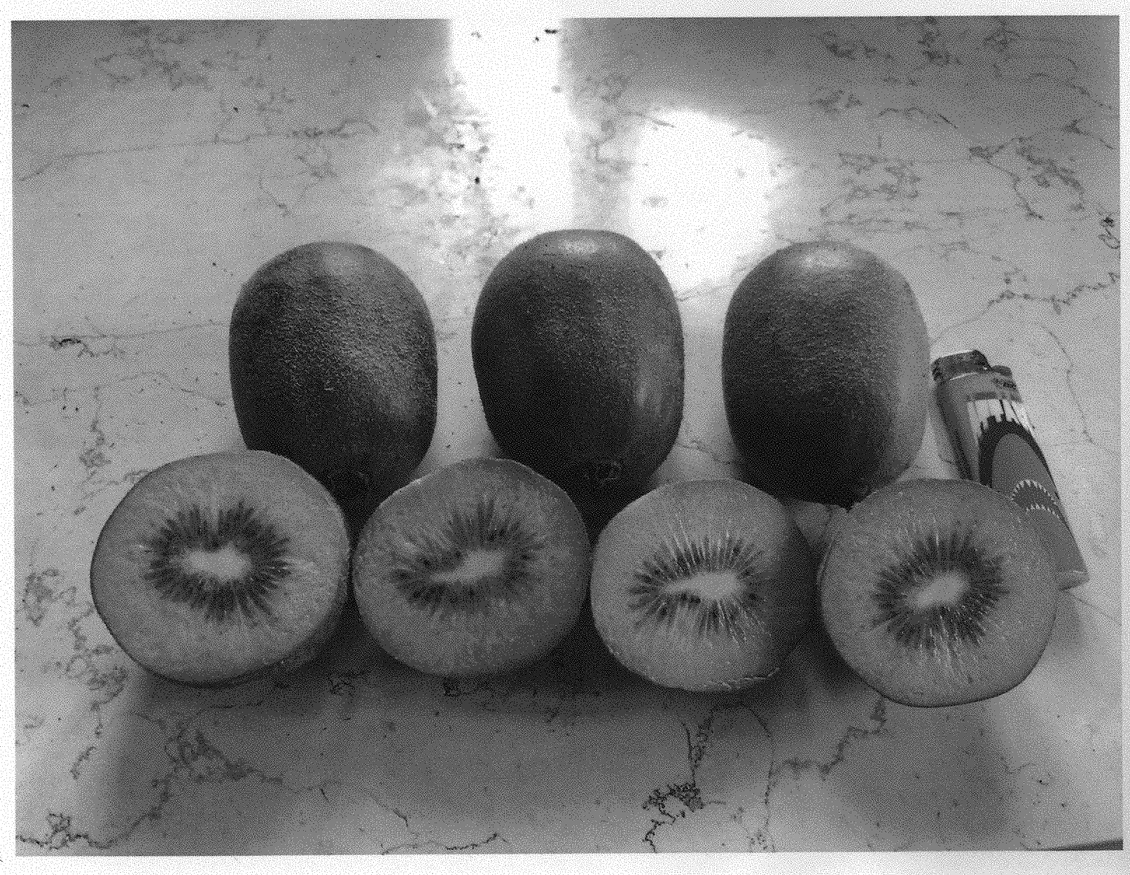

FIG. 5 shows a typical fruit of the new variety in the studio and on a scale.

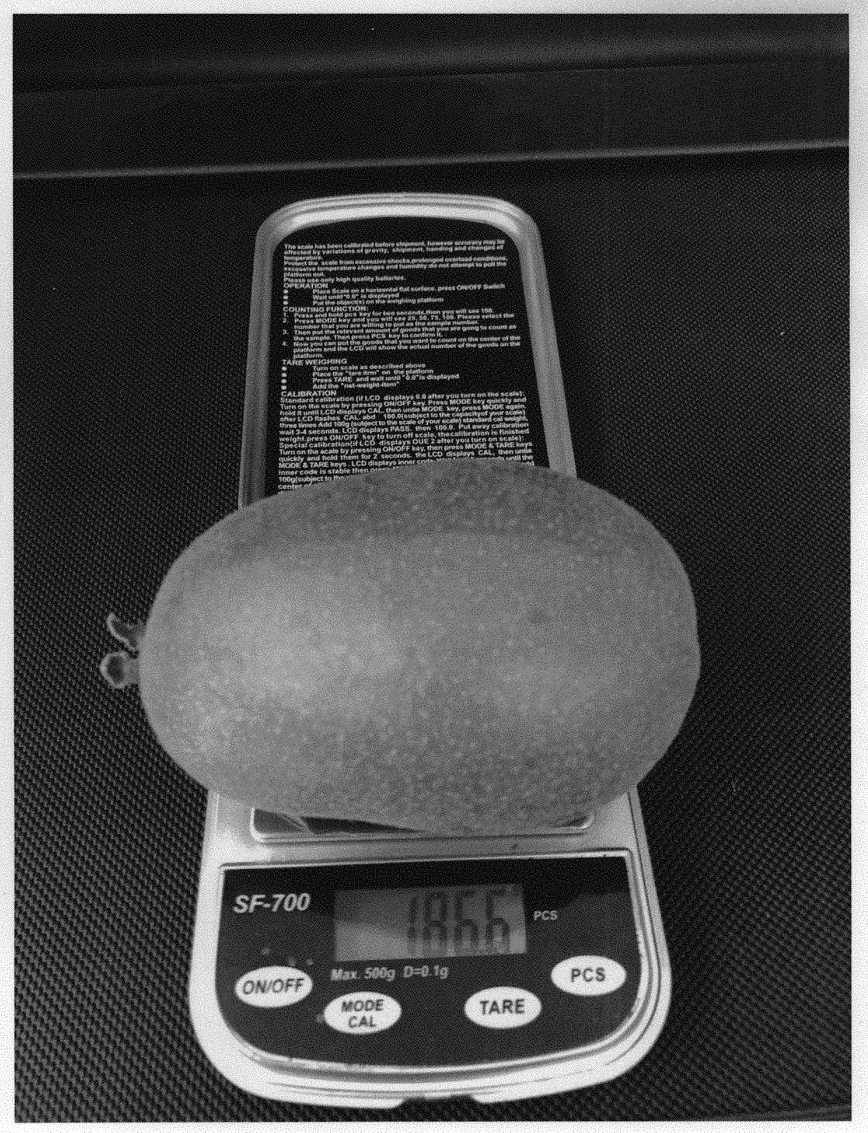

FIG. 6 shows typical fruit of the parent female RS 1 species in the studio, including in cross-section.

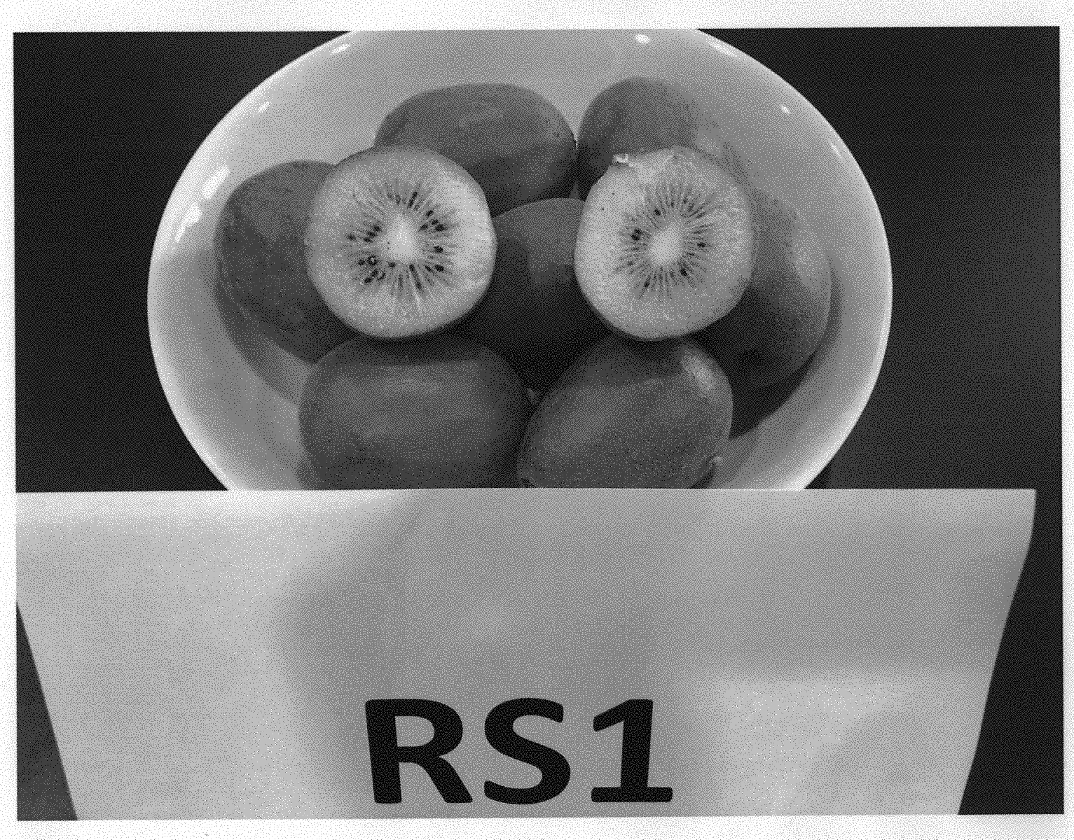

FIG. 7 shows typical fruit of the new variety in the studio compared with the parent female RS1 species, including in cross-section.

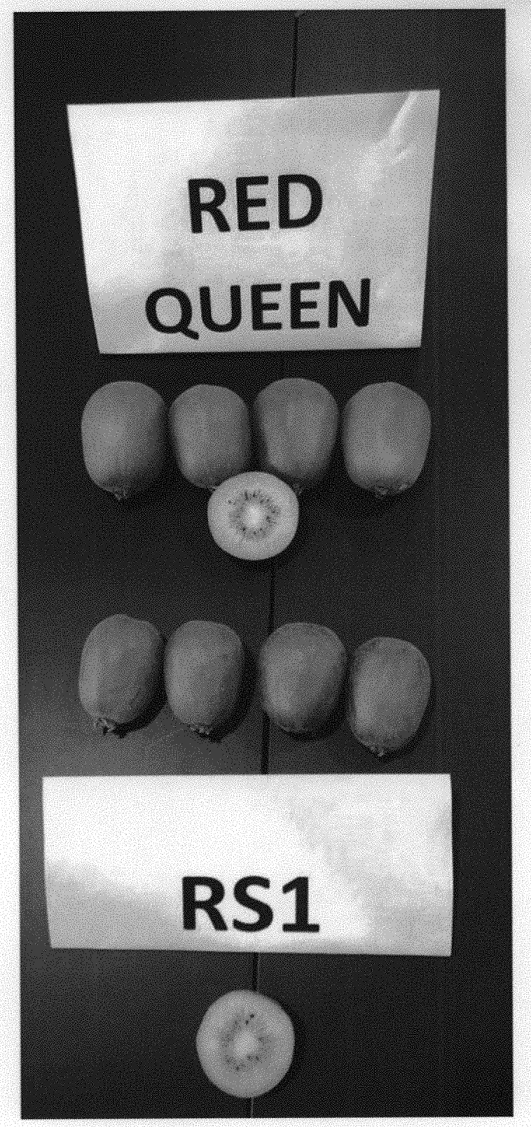

### COMPARISON TO CLOSEST VARIETY

RK 2018 variety shows some distinctive features common to RS1 varieties, such as a navel at the bottom, a slightly pear shape, hairless fruits and very high fertility.

Comparison with the similar variety RS1 (protected by CPVO Europe under the title # EU46694) shows that RK 2018 may be distinguished as follows in Table 2:

Comparison With Similar Variety.
Observations made under Italy Growing Conditions 2016-2019

| Characteristic | RS1 | | RK 2018 | |
|---|---|---|---|---|
| FRUIT: | | | | |
| Harvest date | Late September | | Late October | |
| Color of ripe | Bright yellow (44A)/ | | Greenish yellow (4B)/ | |
| pericarp* | Bright red (44B) | | Bright red (44B) | |
| Skin color | Greenish brown | | Greenish brown | |
| | (152D) | | (152D) | |
| Mean fresh weight | 70-80 | grams | 85-100 | grams |
| Mean dry matter at | 19-23% | | 19-22% | |
| harvest | | | | |
| Average length | 60 | mm | 70 | mm |
| Average width | 37 | mm | 40 | mm |
| Core diameter | 12 | mm | 13 | mm |
| Width/length ratio | 0.61 | | 0.57 | |
| Sweetness (Brix at | 20-22° | | 19-21° | |
| maturity for | | | | |
| consumption) | | | | |
| General shape | Oblong | | Oblong | |
| Cross-sectional shape | Circular | | Circular | |
| Shape of stylar end | Navel | | Navel | |
| Skin: Hairness | Absent | | Almost absent | |
| VINE: | | | | |
| Shoots: | | | | |
| Color | 148A | | 148A | |
| Texture | Smooth | | Smooth | |
| Stem: | | | | |
| Color (upper) | 165A | | 166A | |
| Color (lower) | 199A | | 199A | |
| Mean diameter | 9 | mm | 9.5 | mm |
| Texture | Smooth | | Smooth | |
| Lenticels (if present) | Present | | Present | |
| Leaf: | | | | |
| Color (upper) | 134A | | 134A | |
| Color (lower) | 134B | | 134C | |
| Shape | Orbiculate | | Orbiculate | |
| Length | 120 | mm | 130 | mm |
| Width | 115 | mm | 120 | mm |
| Apex | Acuminate | | Acuminate | |
| Base | Cordate | | Cordate | |
| Margin | Ciliate | | Ciliate | |
| Texture | Smooth | | Smooth | |
| FLOWER: | | | | |
| Inflorescence: | | | | |
| Predominant number | 5-6 | | 4-7 | |
| of flowers | | | | |
| Petiole: | | | | |
| Length | 100-105 | mm | 105-115 | mm |
| Color | 162A | | 162B | |
| Pedicel Length | 35 | mm | 37 | mm |
| Color | 135C | | 135C | |
| Hairs | Smooth | | Smooth | |
| Length of hairs | — | | — | |
| Flower: | | | | |
| Coloration of petals | Bi-colored | | Bi-colored | |
| Primary color | 155A | | 155B | |
| Secondary color | 145A | | 145B | |
| Base of petals | 48 | mm | 50 | mm |
| diameter | | | | |
| Arrangement of | Overlapping | | Overlapping | |
| petals | | | | |
| Mean number of | 5-6 | | 6 | |
| petals/flowers | | | | |
| Mean length of petals | 24 | mm | 26 | mm |
| Mean width of petals | 21 | mm | 22 | mm |
| Petal ratio of | 1.15 | | 1.17 | |
| length/width | | | | |
| Petal shoulder | Present | | Present | |
| Filament color | 157A | | 157A | |
| Anthere color | 6A | | 6B | |
| Attitude of styles | Erect | | Erect | |
| Curvature of styles | Absent | | Absent | |
| Color of styles | 157A | | 157C | |
| Amount of hair on | Dense | | Dense | |
| ovary | | | | |
| Color of ovary | 157A | | 157A | |
| Number of sepals | 6-7 | | 6-7 | |
| Color of sepals | 146C | | 146D | |
| Length of Sepals: | | | | |
| Range | 8.5-11.0 | mm | 9.3-11.5 | mm |
| Mean length | 10.0 | mm | 10.3 | mm |
| Sepals diameter | 6.7 | mm | 7.0 | mm |

| | | |
|---|---|---|
| Flower opening | Mid-April | Late April |
| Vegetative bud break | Mid-March | Late March |
| Plant/fruit disease | None | None |
| and pest resistance | | |
| Plant hardiness zone | Unknown | Unknown |
| or heat/cold | | |
| resistance | | |

*Royal Horticultural Society Color Chart 2001

The most striking differences between RS1 and RK 2018 are the fruit shape (RS1 is more pear-shaped, while RK 2018 is more cylindric shape) and size, flowering and harvest times, and storability. The fruit size is significantly bigger than RS1, and the biggest fruits on RK 2018 are obtained on medium wood, while the RSI needs larger, strong wood to reach decent size. The bud break and blooming of RK 2018 are both delayed by about 1 week beyond that of RS1. Also, fruit storability or RK 2018 is much longer than that of RS1, typically 4-5 months for RK 2018 versus two months for RS1.

The embodiments of the invention in which an exclusive property or privilege is claimed are defined as follows:

1. A new and distinct kiwi plant of the species A. Chinensis Planch substantially as herein described and illustrated.
